(19) United States
(12) Patent Application Publication
(10) Pub. No.: US 2007/0225639 A1
(43) Pub. Date: Sep. 27, 2007

(54) Supporting device for containers of biomedical fluids for parenteral administering, particularly bottles and phials for medical fluids, blood or the like

(75) Inventor: Graziano Azzolini, Cavezzo (MO) (IT)

Correspondence Address: BROWDY AND NEIMARK, P.L.L.C.;624 NINTH STREET, NW, SUITE 300, WASHINGTON, DC, 20001-5303, US

(73) Assignee: Sidam di Azzolini Graziano e c.S.a.s., Giacomo Roncole Mirandola (MO) (IT)

(21) Appl. No.: 11/727,409

(22) Filed: Mar. 26, 2007

(30) Foreign Application Priority Data
Mar. 24, 2006 (IT) MO 2006 A 000098

(51) Int. Cl. A61M 5/14

(52) U.S. Cl. 604/80

(57) ABSTRACT

The supporting device for containers of biomedical fluids for parenteral administering, particularly bottles and phials for medical fluids, blood or the like, comprises an element substantially shaped like a cup, having an upper fitting opening for fitting a container for biomedical fluids, a lower exit opening of the fluid, an outer surface made of heat insulating material and an inner surface suitable for facing the container and of which at least one portion is associable with heat energy conduction and accumulation means.

The present invention refers to a supporting device for containers of biomedical fluids for parenteral administering, particularly bottles and phials for medical fluids, blood or the like.

It is a known fact that people with health problems, struck by indispositions and accident victims require medical care and attention that sometimes calls for the parenteral administering of biomedical fluids and/or liquids (drugs, physiological salt solutions, blood or the like) at a pre-established temperature. In some cases for example, patients remain in a state of shock characterised by hypovolemia (volumetric reduction of the blood) and hypothermia (drop in body heat), and fluids have to be infused at a temperature generally between 33° C. and 40° C.

For the treatment of tumoral illnesses, on the other hand, the need often arises to administer chemotherapy products the effectiveness of which varies according to temperature and which, therefore, have to be infused at a temperature generally above room temperature.

Similar considerations can be made as regards the means of contrast used in diagnostics, these too commonly administered at the patient's body temperature. In conditions of brain distress, on the other hand, it is often best to reduce the body temperature of the patient by means of the infusion of cooled fluids.

The fluids to be injected are currently packaged in containers, of the flexible bag, plastic phial or glass bottle type, that have a dispensing mouth from where the fluid can be taken by means of a standard syringe or by means of a sampling and transport duct, commonly called a “defluxion device”, the exit end part of which is associated with a patient injection element (catheter or needle).

To condition the temperature of the fluids, use is normally made of particular systems for heating and/or cooling the fluid containers and/or the fluid sampling and transport duct.

To cool the fluid to be infused, for example, use is usually made of iced water containers, in which the sampling and transport duct is manually immerged by a health operator every time the need is felt; in such cases however, the control of the temperature of the fluid at the time of the infusion is absolutely subjective and uncertain and risks jeopardising the effectiveness of the treatment and/or the health of the patient unless performed by expert and professional medical staff.

To heat the fluid, on the other hand, the use is known of devices essentially made up of a box inside which the containers are placed connected to the patient by means of the defluxion devices and which, during the dispensing of the fluid, is heated by heat production means, e.g. of the electric resistor type; such box also has an electronic temperature control and adjustment circuit.

Other devices of known type heat the sampling and transport duct during administering and essentially consist of a metal element which is coupled with electric resistors, supplied with electricity, and which is placed close to the portion of the defluxion device to be conditioned; in this case as well an electronic circuit controls and adjusts the achieved temperature.

These devices for heating the container and/or the sampling and transport duct have a number of drawbacks among which we can recall the fact that they are of considerable weight and overall dimensions that makes transport and use particularly difficult and laborious, that they are complex in terms of structure and construction, that they require the intervention of skilled operators and are fairly costly.

Another drawback of these known heating devices consists in the fact that, using electricity, they can create interferences with other electric/electronic devices located near the patient.

Devices are also known for heating or cooling the fluid containers which, in jargon, are defined as being of the so-called “chemical” type because they exploit the heat produced by an exothermic or endothermic chemical reaction between two or more reacting substances.

These devices of “chemical” type are essentially composed of a wrapping, of the bag type, split into two or more compartments separated the one from the other by a tearable membrane and of which one contains a first component and the other contains a second component suitable for reacting together with an exothermic or endothermic reaction depending on the type of intended use of such devices.

At the time of use, an operator acts manually on the wrapping, exercising on this a pressure such as to break the tearable membrane and allow the chemical combination of the components, and then manually places the device in contact with the container of the fluid to be administered to thermally condition it. These “chemical” type devices also have a number of drawbacks such as, for example, the fact that they have to be stored, transported and handled with care and attention to prevent suffering accidental knocks that involuntarily and at unsuitable times activate the reaction between the chemical components they contain, with the risk of prematurely exhausting their capacity to develop calories/refrigeration units and, therefore, become unusable at the time of actual need.

Another drawback of the “chemical” type devices consists in the fact that they contain air which, because it has a low heat conduction, acts as insulation and reduces the exchange of heat with the containers of the fluids to be conditioned. As an alternative to the previously described systems and devices, the use is known of special heating appliances, or cooling appliances, composed of an electric oven, or a cool cell, inside which the containers are placed to keep the fluid at the desired temperature and from which they are removed only an instant before being used.

The use of such appliances, though very easy and functional, nevertheless permits administering the fluid at the required temperature only for a limited period of time because, once taken out of the ovens, or of the cool cells, the containers abruptly start to exchange heat with the outside and quickly tend to reach room temperature.

The main aim of the present invention is to provide a supporting device for containers of biomedical fluids for parenteral administering, particularly bottles and phials for medical fluids, blood or the like, that favours the infusion to patients of fluids brought to a pre-established temperature without requiring, during administering, complicated and cumbersome heating/cooling systems/devices.

A further object of the present invention is to provide a device that can be used in conditions of utmost safety for the patient and maximum efficiency of use.

Another object of the present invention is that it is simple in terms of structure and construction, has limited weight and overall dimensions and is easy to transport and handle including by inexpert health operators.

The above-indicated objects are all reached by the present supporting device for containers of biomedical fluids for parenteral administering, particularly bottles and phials for medical fluids, blood or the like, comprising at least one element substantially shaped like a cup, having at least one upper fitting opening for fitting a container for biomedical fluids and at least one lower exit opening of the fluid, wherein said cup element comprises at least one outer surface made of heat insulating material and at least one inner surface suitable for facing said container and of which at least one portion is associable with heat energy conduction and accumulation means.

Further characteristics and advantages of this invention will appear even more evident from the detailed description of a preferred, but not exclusive, embodiment of a supporting device for containers of biomedical fluids for parenteral administering, particularly bottles and phials for medical fluids, blood or the like, illustrated indicatively by way of non limiting example, in the attached drawings wherein:

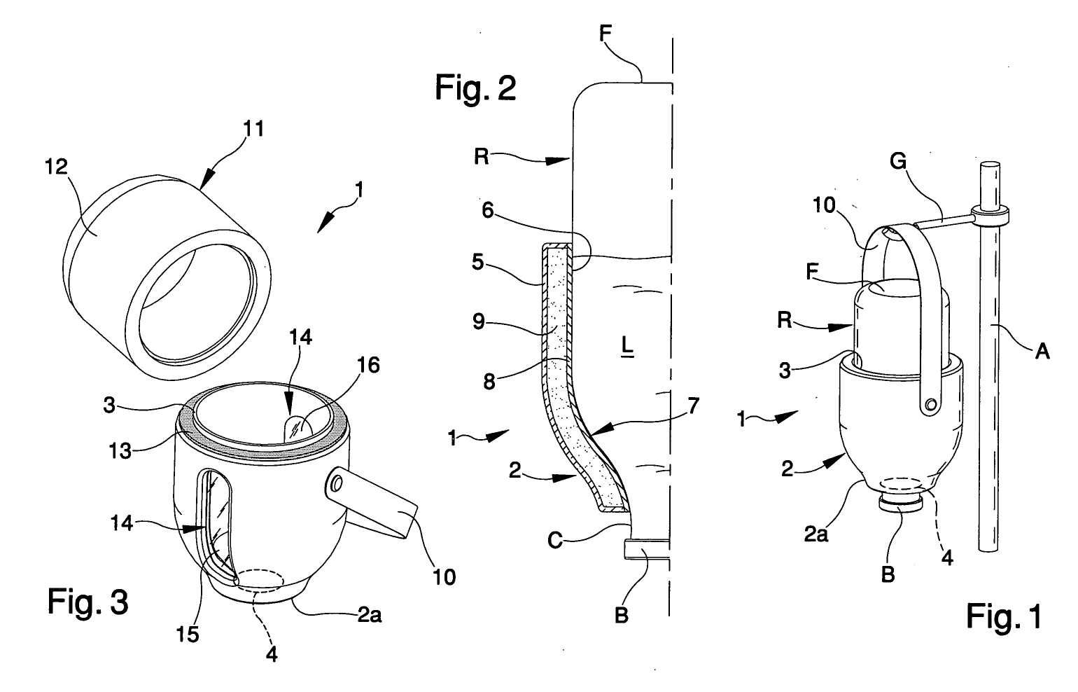

FIG. 1 is an axonometric view of a first embodiment of the device according to the invention, used for supporting a bottle containing medical fluids;

FIG. 2 is a schematic and partial section view of the device of FIG. 1;

FIG. 3 is an axonometric view of a second embodiment of the device according to the invention;

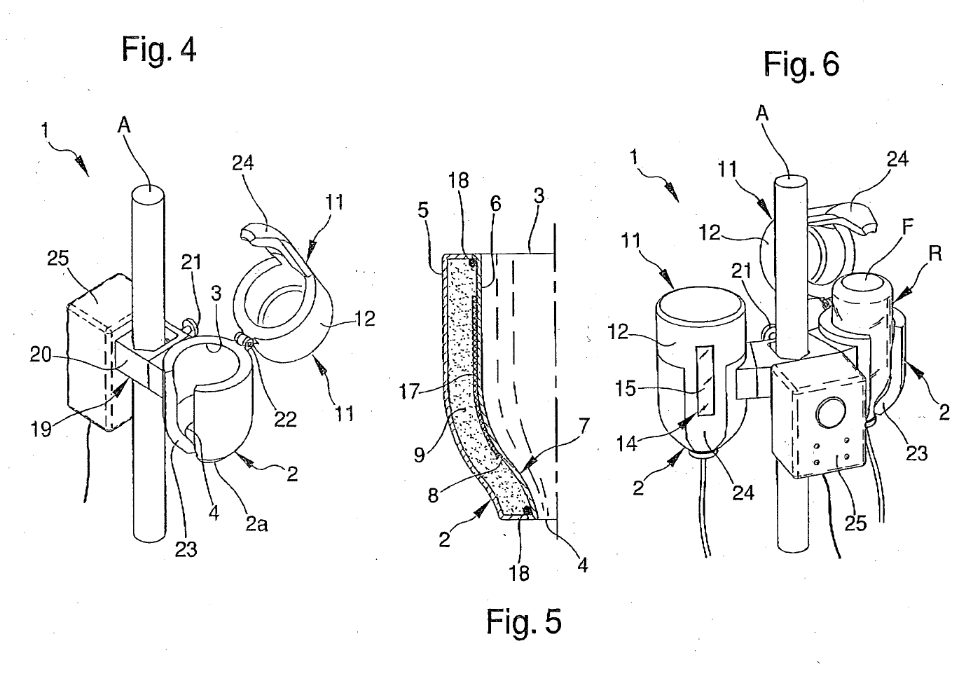

FIG. 4 is an axonometric view of a further embodiment of the device according to the invention;

FIG. 5 is a schematic and partial section view of the device of FIG. 4;

FIG. 6 is an axonometric view of the embodiment of FIG. 4 with two cup elements.

With particular reference to such figures, a supporting device has been globally indicated by 1 for containers of biomedical fluids for parenteral administering, particularly bottles and phials for medical fluids, blood or the like.

In the particular embodiments shown in the figures, the container R consists of a rigid bottle for containing the fluid L to be injected in a patient for diagnostic purpose according to the diagnostic techniques; such bottle has a neck C converging and terminating in a reclosable dispensing mouth B.

It cannot however be ruled out that the present invention can also be used with flexible bags, semi-rigid phials or other type of containers for biomedical use, containing blood, drugs or other substances to be administered.

The device 1 comprises a substantially rigid cup element 2 having, at the top, a fitting opening 3 for fitting the container R and, in the lower portion, a fluid exit opening 4.

The cup element 2 comprises an outer surface 5 made of heat insulating material, e.g. of the polymeric type, and an inner surface 6 which, during use, is suitable for being arranged facing the container R and which is associated with heat energy conduction and accumulation means 7.

Such conduction and accumulation means are composed of a first layer 8 made of heat conductive material, which defines the inner surface 6 of the cup element 2, and of a second layer 9 made of heat insulating material, which is suitable for accumulating heat energy (calories or refrigeration units) and substantially placed in between the outer surface 5 and the inner surface 6.

The heat conductive material of the first layer 8 is of the metal type, e.g. aluminium.

The outer surface 5 and the first layer 8 define between them an interspace insulated from the outside and completely filled with the heat insulating material of the second layer 9 which, advantageously, is in the fluid state (air or liquid).

The cup element 2 has a cross section substantially circular in shape and a lower portion 2a shaped like a funnel converging downwards, on which rests the neck C of the container R once this has been fitted through the fitting opening 3.

In use configuration, the dispensing mouth B of the container R protrudes from the exit opening 4.

The fitting opening 3 has substantially larger dimensions than the exit opening 4; the fitting opening 3, as a matter of fact, is defined by the upper open edge of the cup element 2, while the exit opening 4 is defined by the lower open edge of the funnel-shaped lower portion 2a.

The inner surface 6 of the cup element 2, in actual fact, is substantially complementary to the shape of the container R, so that, during use, the inner surface 6 of the cup element 2 and the outer surface of the container R are placed in reciprocal contact of the conforming type.

Alternative embodiments of the present invention cannot, however, be ruled out where the dimensions of the cup element 2 are substantially larger than the container R, in this case dimension adapting means can be usefully provided, suitable for transmitting heat between the cup element 2 and the container R without the interposition of air bags.

Such adapting means, not shown in detail in the figures, can consist, for instance, of an additional tubular shaped body, made of heat conductive material and having an outer side complementary to the inner surface 6 of the cup element 2 and which can be placed exactly in contact with this, and of an inner side complementary to the container R and which can be placed exactly in contact with this.

The heat conductive material of the additional body, when provided, can usefully coincide with the heat conductive material of the first layer 8 and be, for example, in aluminium.

Close to the fitting opening 3 are provided coupling means 10 of the cup element 2 to a supporting hook G, e.g. of the type of those at the top of the traditional drip support rods A commonly used in hospitals and medical clinics. In detail, the coupling means 10 are defined by a brace which is associated with diametrically opposite parts of the outer surface 5 of the cup element 2 and can be hung on the supporting hook G.

FIGS. 1 and 2 show a first embodiment of the invention wherein the height of the cup element 2 is such that, in use configuration, the bottom F of the container R remains outside the element itself.

In consideration of the fact that, in use configuration, the fluid L to be administered collects near the dispensing mouth B and the neck C of the container R, it can easily be understood how the cup element 2 is able to exchange heat energy with the part of the container R directly in contact with the fluid L, allowing this to be heated/cooled in an efficient way.

In a second embodiment of the invention shown in FIG. 3, on the other hand, the device 1 is fitted with closing means 11 for closing the fitting opening 3 which are of the removable type, i.e. they can be fitted once the container R has been introduced into the cup element 2 and removed when the container R needs to be pulled out.

In detail the closing means 11 consist of a cover 12 shaped so as to define a semi-shell which, in operating configuration, is suitable for covering the portion of the container R protruding from the opening itself, so as to completely heat insulate the container R from the external environment.

The open edge of such cover 12, in detail, is associable with the edge of the fitting opening 3 by interposition of sealing means 13, of the type of a ring-shaped seal fixed to the edge of the fitting opening 3; it should be noted how, in closing configuration, such sealing means prevent the passage of air, and therefore heat, between the external environment and the inside of the device 1. Unlike the embodiment shown in FIGS. 1 and 2, in the second embodiment the device 1 is fitted with sight inspection means 14 for inspecting the level of the fluid L inside the container R.

Such sight inspection means comprise a transparent window 15, elongated vertically, which is obtained through a side of the cup element 2.

In consideration of the fact that most fluids used in the biomedical field are transparent and colourless, in order to increase the visibility through the window 15, the sight inspection means 14 also comprise a reflecting body 16, of the mirror type or the like, associated with the inner surface 6 of the cup element 2 at the diametrically opposite part with respect to the window 15.

The present invention according to the embodiments of figures from 1 to 3 operates as follows.

The container R and the device 1 are initially heated, or cooled, by means of a known conditioning apparatus, e.g. inside an electric oven, a cool cell or the like.

During this initial phase of heat conditioning, the container R and the device 1 are left separate the one from the other, allowing the container R to reach the temperature required for administering to the patient and the cup element 2 to accumulate calories, or refrigeration units, inside the first and second layer 8 and 9.

Afterwards the cup element 2 is removed from the electric oven/cool cell together with the container R, is hung to the supporting hook G and the container R is fitted inside through the fitting opening 3.

When provided, the cover 12 can therefore be fitted to the cup element 2 to completely insulate the container R from the external environment.

During administering to patient, the quantity of biomedical fluid L which remains inside the container R tends to exchange heat with the external environment but with a very limited temperature gradient since the heat energy initially accumulated by the first and second layer 8 and 9 is progressively transferred to the container R which maintains the temperature required for administering to the patient for a long time.

In the figures from 4 to 6 a further embodiment of the invention is shown, wherein, conveniently, heating means 17 are arranged inside a cup element 2 (FIG. 4) or two cup elements 2 (FIG. 6).

The heating means 17 consist, e.g., of a electric resistor arranged in contact with the inner side of the first layer 8.

At the axial end parts of the first layer 8, on the joining areas between the first layer and the outer surface 5, are arranged packing means 18, of the type of a pair of rubber O-rings, suitable for preventing any fluid from flowing inside the cup element 2 and, in particular, the contrast liquid L in case of accidental breaking of the container R.

The heating means 17, in actual fact, allow heating the first layer 8 without having to place the cup element or cup elements 2 inside an electric oven or the like.

This feature allows fitting the device 1 with gripping means 19 associated with the cup element 2 and suitable for allowing their mounting, in a firm and rigid way, on the vertical upright of the drip support rod A.

The gripping means 19 are of the clamp type and comprise a block 20, associated integral with the cup element 2 and which can be positioned around the drip support rod A, and screw means 21 which are engaged through the block 20 and suitable for pressing against the rod A.

In the embodiment of the figures from 4 to 6, the device 1 has a cover 12 for each cup element 2 but, unlike the embodiment of FIG. 3, hinging means 22 are provided for hinging the cover itself to the edge of the fitting opening 3.

Furthermore, each cup element 2 has a longitudinal slot 23 which extends from one side to the other between the fitting opening 3 and the exit opening 4.

In the closing configuration, the space defined by the longitudinal slot 23 is intended to be filled by an elongated complementary wall 24, which extends from the cover 12 and on which the sight inspection window 15 is obtained.

Usefully, the operation of the heating means 17 is controlled by means of a central processing and control unit 25 which is associated with the cup element/s 2 by interposition of the block 20.

The present invention according to the embodiment of the figures from 4 to 6 operates as follows.

The central processing and control unit 25, in actual fact, allows controlling the temperature of the first layer 8 and interrupting the power supply to the electric resistor 17 once verified that a pre-established temperature has been reached.

When the first and second layers 8 and 9 have accumulated enough heat, the cup element/s 2 is/are able to maintain the temperature of the biomedical fluid L inside the container R for a long time.

Furthermore, in the particular case of the device 1 having two cup elements 2 (FIG. 6), the central processing and control unit 25 is able to control the alternate heating of the cup elements 2.

By so doing, while one cup element 2 is in the heating phase by means of its own heating element, the other cup element 2 has already accumulated enough heat and is used for administering the biomedical fluid L to the patient.

This makes it possible to always have at disposal at least one cup element 2 the conduction and accumulation means 7 of which are warm and ready for use.

It has in fact been found that the described invention achieves the proposed objects.

The invention thus conceived is susceptible of numerous modifications and variations, all of which falling within the scope of the inventive concept.

Furthermore, all the details may be replaced by other elements which are technically equivalent.

In practice, the materials used, as well as the contingent shapes and dimensions, may be any according to requirements without because of this moving outside the protection scope of the following claims.

1) Supporting device for containers of biomedical fluids for parenteral administering, particularly bottles and phials for medical fluids, blood or the like, comprising at least one element substantially shaped like a cup, having at least one upper fitting opening for fitting a container for biomedical fluids and at least one lower exit opening of the fluid, wherein said cup element comprises at least one outer surface made of heat insulating material and at least one inner surface suitable for facing said container and of which at least one portion is associable with heat energy conduction and accumulation means.

2) Device according to claim 1, wherein said conduction and accumulation means comprise at least one first layer made of heat conductive material.

3) Device according to claim 1, wherein said conduction and accumulation means comprise at least one second layer made of heat insulating material substantially placed in between said outer surface and said portion of inner surface.

4) Device according to claim 1, wherein said cup element is rigid.

5) Device according to claim 1, wherein said heat insulating material of said outer surface is substantially of the polymeric type.

6) Device according to claim 2, wherein the heat conductive material of said first layer is of the metal type.

7) Device according to claim 6, wherein said heat conductive material of metal type is aluminium.

8) Device according to claim 2, wherein said inner surface is defined by said first layer.

9) Device according to claim 3, wherein the heat insulating material of said second layer is in the fluid state.

10) Device according to claim 1, wherein said inner surface is substantially complementary to the shape of said container and suitable for being placed in contact with said container.

11) Device according to claim 1, wherein the dimensions of said cup element are substantially larger than said container and in that it comprises dimension adapting means suitable for transmitting heat between said inner surface and said container.

12) Device according to claim 11, wherein said adapting means comprise at least one additional body, substantially tubular in shape, at least partially made of heat conductive material and having an outer side, substantially complementary to said inner surface and which can be placed in contact with this, and an inner side, substantially complementary to said container and which can be placed in contact with this.

13) Device according to claim 1, wherein it comprises closing means for closing said fitting opening.

14) Device according to claim 13, wherein said closing means are of the removable type.

15) Device according to claim 13, wherein said closing means comprise at least one cover associable with the edge of said fitting opening.

16) Device according to claim 15, wherein said cover comprises a semi-shell for covering the portion of said container protruding from said fitting opening.

17) Device according to claim 15, wherein said closing means comprise sealing means which can be placed in between said cup element and said cover.

18) Device according to claim 17, wherein said sealing means comprise at least one ring-shaped seal associated with the edge of said fitting opening.

19) Device according to claim 15, wherein said closing means comprise hinging means for hinging said cover to said cup element.

20) Device according to claim 1, wherein it comprises sight inspection means for inspecting the level of said biomedical fluid inside said container.

21) Device according to claim 20, wherein said sight inspection means comprise at least one window made of substantially transparent material which is obtained through a side of said cup element.

22) Device according to claim 21, wherein said window is elongated in a substantially vertical direction.

23) Device according to claim 21, wherein said sight inspection means comprise at least one reflecting body associated with said inner surface at the diametrically opposite part with respect to said window.

24) Device according to claim 1, wherein it comprises heating means of said conduction and accumulation means, which are associated with said cup element.

25) Device according to claim 24, wherein said heating means are substantially placed in between said first layer and said second layer.

26) Device according to claim 24, wherein said heating means comprise at least one electric resistor arranged in contact with the inner side of said first layer.

27) Device according to claim 26, wherein it comprises packing means suitable for preventing any fluid from flowing inside said cup element.

28) Device according to claim 24, wherein it comprises at least one central processing and control unit associated with said cup element and with said heating means and suitable for controlling the heating of said conduction and accumulation means.

29) Device according to claim 1, wherein it comprises gripping means associated with said cup element and suitable for allowing their mounting on a vertical upright.

30) Device according to claim 29, wherein said gripping means are of the clamp type.

31) Device according to claim 1, wherein it comprises two of said cup elements associated between them.
